(19) United States
(12) Patent Application Publication
(10) Pub. No.: US 2020/0077986 A1
(43) Pub. Date: Mar. 12, 2020

(54) ANGLES FOR ULTRASOUND-BASED SHEAR WAVE IMAGING

(71) Applicant: Siemens Medical Solutions USA, Inc., Malvern, PA (US)

(72) Inventor: Yassin Labyed, Maple Valley, WA (US)

(21) Appl. No.: 16/128,801

(22) Filed: Sep. 12, 2018

(51) Int. Cl. A61B 8/08; A61B 8/00

(52) U.S. Cl. CPC A61B 8/5223; A61B 8/085; A61B 8/14; A61B 8/469; A61B 8/463; A61B 8/485

(57) ABSTRACT

For shear wave imaging with ultrasound, a direction of the ARFI beam is selected based on tissue information, such as being perpendicular to an orientation of tissue or other than perpendicular to a face of the transducer array. As a result, the estimated shear wave velocity measured perpendicular to the ARFI beam may be closer to actual shear wave velocity. Alternatively or additionally, one or more vectors of propagation of the shear wave are determined and displayed to the user, allowing the user to visualize an extent of anisotropy of the tissue to judge impact on the shear wave velocity estimation.

### BACKGROUND

The present embodiments relate to shear wave imaging. Shear wave speed in tissue may be diagnostically useful, so ultrasound is used to estimate the shear speed in a patient's tissue. By transmitting an acoustic radiation force impulse (ARFI) along a transmit scan line near or in a region of interest, a shear wave is generated at the ARFI focus. The shear wave is assumed to predominantly propagate perpendicular to the transmit scan line. Ultrasound scanning monitors the propagation of the shear wave within the region of interest. The arrival time of the shear wave at a distance from the origin of the shear wave is used to determine the velocity of the shear wave in the tissue. The speed for different locations within the region of interest may be estimated, providing a spatial distribution of shear wave velocity.

Anisotropic tissue may impact shear wave generation, propagation, and detection. Muscle, collagen, or other fibers may cause the shear wave to predominantly propagate at a different angle than perpendicular to the transmit beam of the ARFI. The assumption that the shear wave propagates perpendicular to the ARFI beam results in underestimation of the shear wave velocity. Ultrasound imaging systems provide no tools for characterizing shear wave anisotropy, so users may alter a field of view to measure shear wave velocity from different viewpoints. This approach is inexact and time consuming.

### SUMMARY

By way of introduction, the preferred embodiments described below include methods, computer readable storage media with instructions, and systems for shear wave imaging with ultrasound. A direction of the ARFI beam is selected based on tissue information, such as being perpendicular to an orientation of tissue or other than perpendicular to a face of the transducer array. As a result, the estimated shear wave velocity measured perpendicular to the ARFI beam may be closer to actual shear wave velocity. Alternatively or additionally, one or more vectors of propagation of the shear wave are determined and displayed to the user, allowing the user to visualize an extent of anisotropy of the tissue to judge impact on the shear wave velocity estimation.

In a first aspect, a method is provided for shear wave imaging with an ultrasound scanner. A region of interest for tissue of a patient is positioned, and an angle is received. A radiation force pulse is transmitted from a transducer of the ultrasound scanner to a focus location in or by the region of interest of the tissue of the patient. The radiation force pulse is transmitted to intersect the focus location at the angle. A shear wave is generated due to the radiation force pulse. The ultrasound scanner scans the region of interest with ultrasound as the shear wave propagates in the region of interest. A shear wave characteristic is estimated from the scanning. An image of the shear wave characteristic of the tissue of the patient is generated.

In a second aspect, a method is provided for shear wave imaging with an ultrasound scanner. A radiation force pulse is transmitted from a transducer of the ultrasound scanner to tissue of a patient. A shear wave is generated due to the radiation force pulse. The ultrasound scanner scans the tissue with ultrasound as the shear wave propagates in the tissue. A direction of propagation of the shear wave is determined from the scanning. An image representing the direction of propagation of the shear wave in the tissue of the patient is generated.

In a third aspect, a system is provided for shear wave imaging with ultrasound. A transmit beamformer is configured to transmit a pushing pulse along a transmit line into tissue of a patient. A transmit angle of the transmit line of the pushing pulse relative to a location in the tissue is selectable. A receive beamformer is configured to receive signals from scanning after the transmission of the pushing pulse. An image processor is configured to determine, from the receive signals, shear wave velocity and a propagation angle of a shear wave in the tissue. A display is configured to output a shear velocity image of the shear wave velocity with a graphic representing the propagation angle.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for shear wave imaging with an ultrasound scanner;

FIG. 2 illustrates an example spatial arrangement for a region of interest and ARFI transmit scan line for shear wave imaging;

FIG. 3 illustrates an example spatial arrangement for a region of interest with an angled ARFI transmit scan line and imaging with propagation vectors; and

FIG. 4 is a block diagram of one embodiment of a system for shear wave imaging.

### DETAILED DESCRIPTION OF THE DRAWINGS AND PRESENTLY PREFERRED EMBODIMENTS

Shear wave vector imaging is provided. Tissue anisotropy causes shear waves to predominantly propagate along preferential directions. Dealing with the anisotropy may improve shear wave elasticity imaging (SWEI) and provide additional clinical benefits. In many ultrasound systems, it is difficult to assess anisotropy because the angles of push and track beams in SWEI are not controlled by the user. Shear wave vector imaging uses the vector for the push beam and/or detected vector of shear wave propagation.

Shear wave vector imaging may use an angle of the push beam to better deal with anisotropy. In conventional SWEI, the angle of the push beam is not controlled but instead is perpendicular to the transducer. The angle of the push beam is selected using a user control or image processing and is independent of region of interest control. By user or automated control of the push beam angle, the resulting estimates of shear wave characteristics may be more accurate.

Shear wave vector imaging may display a vector or vectors showing shear wave propagation magnitude and/or direction. By determining shear wave characteristic along the propagation direction, the estimate may be more accurate. The indication of the vector direction may assist the user in diagnosis and/or to determine the accuracy or likely error of the SWEI. The vector is displayed independently of and/or overlaid on the shear wave velocity or displacement color map. In one embodiment, a gradient of an arrival time map is computed to obtain a shear wave velocity as a vector field. In another embodiment, displacement maps from tracking beams at different angles are combined to compute magnitude and direction of a shear wave displacement field.

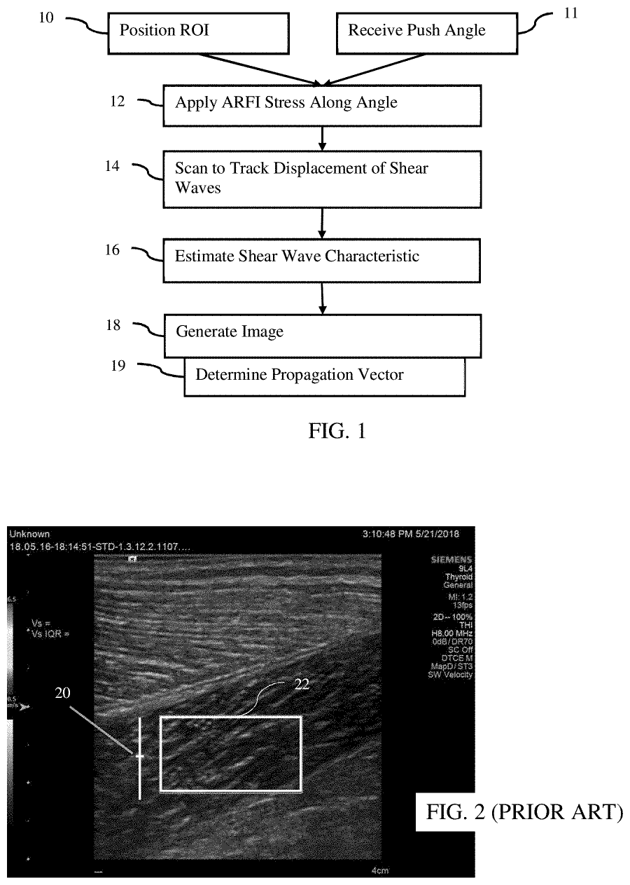

FIG. 1 shows one embodiment of a method for shear wave imaging with an ultrasound scanner. The angle of the ARFI beam is selectable, such as based on tissue anisotropy. The angle of propagation of the shear wave may be determined and displayed. Either one or both of the angle of the ARFI beam and detected angle of propagation may be used.

The method is implemented by the system of FIG. 4 or a different system. A controller, user interface, and/or image processor receive the push angle in act 11 and/or positioning of the region of interest in act 10. Transmit and receive beamformers use a transducer to transmit and receive from the patient, including applying ARFI at a selectable angle and tracking the tissue response in acts 12 and 14. An image processor estimates the shear wave characteristic in act 16. The image processor generates the image in act 18. A display may be used for act 18. Different devices, such as other parts of an ultrasound scanner, may perform any of the acts.

The acts are performed in the order described or shown (i.e., top to bottom), but may be performed in other orders. Acts 10 and 11 may be performed in any order or may be performed simultaneously. Act 19 may be performed prior to act 18 and/or act 16.

Additional, different, or fewer acts may be provided. For example, act 11 or act 19 is not performed. Acts for configuring the ultrasound scanner, positioning the transducer, and/or recording results may be provided. In another example, reference scanning is performed prior to act 12, such as B-mode scanning to detect tissue anisotropy. To determine tissue motion caused by shear waves, the tissue in a relaxed state or subject to no or relatively little shear wave is detected as a reference. The ultrasound scanner detects reference tissue information. The reference scanning occurs prior to transmission of the ARFI in act 12 but may be performed at other times. Any type of detection may be used, such as a B-mode detection of the intensity. In other embodiments, the beamformed data without detection is used as the reference.

In act 10, the ultrasound scanner (e.g., user interface, controller or image processor) of the ultrasound scanner positions a region of interest for tissue of a patient. After scanning the patient, a B-mode or other image is generated. The user inputs using a user input device a region of interest on the image, such as selecting a point about which the two or three-dimensional region is placed. Alternatively, the image processor detects a location for placement of the region of interest, such as applying a machine-learned detector to identify tissue to be measured for a shear wave characteristic.

The region of interest is positioned by selection of a point, placement of an area, or placement of a volume. The region of interest has any shape, such as from tracing a tissue region. In one embodiment, the region of interest is rectangular or square, so the user selects diagonal corner locations or a point and sizing.

In act 11, the ultrasound scanner receives an angle. The received angle is perpendicular to the orientation of the tissue. The angle is based on an orientation of anatomy or expected propagation direction of a shear wave. The angle is set due to the anisotropic direction of the tissue in the region of interest. For example, the direction of fibers (e.g., muscle or collagen) provides an orientation. Where the fibers have different orientations within the region of interest, a median, average, or predominant orientation is used. The angle is perpendicular to the orientation. The angle is used for orientating the push or ARFI beam.

Alternatively, the received angle is the orientation of the tissue. The angle of tissue may be used to determine a perpendicular angle to the expected propagation.

The ultrasound scanner receives the angle as a user input with an input device on the displayed image. The user places a vector, such as entering beginning and ending points. FIG. 2 shows a region of interest 22 as a rectangular region placed over tissue represented in a B-mode image. The sides of the region of interest 22 are parallel with the image (e.g., are horizontal and vertical), but may be tilted or at other angles. Other shapes may be used. Within the region of interest, the tissue is muscle fibers. The fibers are generally oriented from lower left to upper right.

The angle is determined independently of the region of interest control. The angle is based on tissue, such as tissue within the region of interest. The angle is not keyed to the orientation of the region of interest but may be. For user input of the angle, the angle is controlled as a separate input, such as sequencing to angle indication after placing the region of interest. The focal location of the ARFI beam may be set at a given position relative to the region of interest, but the angle is controlled or selected independently of setting the region of interest position.

In FIG. 2, the transmit scan line 20 is represented by a vertical line with a horizontal line for the focal depth. The control to place the region of interest 22 automatically positions the transmit scan line 20 a given distance from a side (either in or out of the region 22). The focal depth is automatically set at a given depth relative to the region of interest.

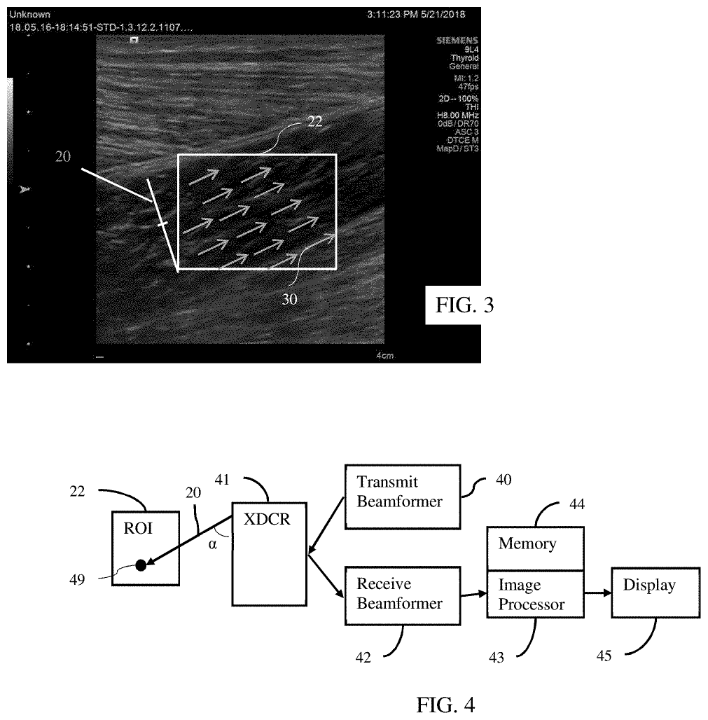

FIG. 3 represents independent control of the angle. While the focal position may be at a same location relative to the region of interest 22, the angle representing the transmit scan line 20 for the ARFI is altered or set to be other than vertical. The angle may be limited by the transducer. The angle is not parallel or perpendicular with the sides of the region of interest 22. The focal depth and/or position may also be selected in other embodiments. The two lines for the transmit scan line 20 and the focal depth may be displayed for the user to position and/or displayed based on determined angle by the ultrasound scanner. Alternatively, the line graphics are not displayed, representing the angle for discussion herein.

Alternatively, the image processor or controller receives the angle as an output of detection by the image processor. Directional filtering, machine-learned detection, or other image processing is used to detect the orientation of the tissue. The angle of the transmit scan line 20 for the ARFI beam is set to be perpendicular to the detected angle of the tissue anisotropy or closer to perpendicular than vertical (e.g., angled to an extent allowed by the transducer while having a sufficiently large aperture to provide the ARFI power). The angle is determined from image processing without user input of the angle. Alternatively, the user inputs a starting angle that is refined by image processing or vice versa.

In one embodiment, the direction of propagation of a shear wave is detected and used to set the angle for subsequent shear wave imaging. Any of the approaches discussed below for act 19 may be used to detect the shear wave propagation direction, such as determining the orientation of propagation and tissue from arrival time of the shear wave or from displacements along non-parallel receive scan lines. The angle of the transmit scan line 20 for the ARFI beam or pushing pulse is set perpendicular to the orientation or direction of propagation caused by the tissue anisotropy.

The transducer may limit the steering angle given a position and size of the region of interest in the field of view. The angle is selected to be away from vertical given the transducer as up or the top or away perpendicular to a center of the transducer or aperture. Perpendicular to the orientation of the tissue may be desired, but closer to perpendicular than parallel with a center of the transducer face and/or transmit may be used.

In act 12, the ultrasound scanner transmits ARFI, and, in act 14, repetitively scans (e.g., transmitting tracking pulses and receiving responsive ultrasound data) the tissue. The repetitive scanning tracks displacements of the tissue caused by a shear wave generated from the transmission of act 12. The shear wave characteristic is estimated in act 16 from the ultrasound data.

In act 12, the ultrasound scanner uses the transducer to apply stress to the tissue. ARFI (i.e., pushing pulse) is transmitted to apply the stress. The ARFI may be generated by a cyclical pulsed waveform of any number of cycles (e.g., tens or hundreds of cycles). For example, ARFI is transmitted as a pushing pulse with 100-1000 cycles. The transmit beamformer generates waveforms for elements of a transmit aperture, and the transducer generates acoustic energy in response to the electrical waveforms. The transmitted acoustic wave propagates along the scan line, causing a deposition of energy and inducing a shear wave. The ARFI is transmitted along the scan line to intersect with the focal location at the angle. The origin and/or angle from the transducer is set by the transmit beamformer to cause the ARFI beam to be formed along a beam at the angle perpendicular to the orientation of the tissue or away from perpendicular to the transducer. For example, the ARFI beam is formed along the scan line 20 of FIG. 3. In this example, the pushing beam is not perpendicular or parallel with any of the sides of the region of interest 22 but is perpendicular to the orientation of the anisotropic tissue. The angle for the ARFI scan line may be away from perpendicular to the orientation of the tissue due to transducer limitations, but is closer to perpendicular to the orientation of the tissue than perpendicular to a center of a face of the transducer array.

ARFI focused at a point or focal region is transmitted. The ARFI beam is formed or transmitted along the transmit scan line 20 at the angle. When ARFI is applied to a focused area, the tissue responds to the applied force by moving. The ARFI creates a shear wave that predominantly propagates laterally through the tissue. Anisotropy of the tissue may cause propagation other than laterally. The shear wave causes displacement of the tissue. At each given spatial location in the region of interest 22 spaced from the focus, this displacement increases and then recovers to zero, resulting in a temporal displacement profile. The tissue properties affect the displacement profile.

In act 14, the ultrasound scanner scans the tissue of the patient in the region of interest. The scanning is repeated any number of times to determine the amount of tissue motion at different locations caused by a shear wave. The detected tissue for each scan is compared to a reference scan of the tissue. The comparison occurs over time with the repetitions to determine displacements due to the passing of the shear wave.

Doppler or B-mode scanning may be used for tracking the tissue responding to the stress. Ultrasound data is received in response to transmissions of ultrasound. The transmissions and receptions are performed for different laterally spaced locations in the region of interest (e.g., over an area or over a volume). A sequence of transmissions and receptions are provided for each spatial location to track over time.

Act 14 occurs after the pushing pulse is applied and while the tissue is responding to the stress. For example, transmission and reception occur after application or change in the stress and before the tissue reaches a relaxed state. Ultrasound imaging may be performed before, during and/or after the stress is applied.

For tracking, the ultrasound scanner transmits a sequence of transmit beams or tracking pulses. A plurality of ultrasound beams is transmitted to the tissue responding to the stress. The scan line or lines used for the tracking transmissions are at the angle or parallel to the ARFI transmit scan line, but non-parallel scan lines may be used for tracking.

The plurality of beams is transmitted in separate transmit events. A transmit event is a contiguous interval where transmissions occur without reception of echoes responsive to the transmission. During the phase of transmitting, there is no receiving. Where a sequence of transmit events is performed, a corresponding sequence of receive events is also performed interleaved with the transmissions. A receive event is performed in response to each transmit event and before the next transmit event.

For a transmit event, one or more transmit beams are formed. The pulses to form the transmit beams are of any number of cycles. Any envelope, type of pulse (e.g., unipolar, bipolar, or sinusoidal) or waveform may be used.

The transducer receives ultrasound echoes in response to each transmit event. The transducer converts the echoes to receive signals, which are receive beamformed into ultrasound data representing one or more spatial locations. The receive scan lines for beamforming are parallel to the ARFI transmit scan line 20 but may be non-parallel. The response of tissue at scan lines for receive beams is detected.

Using reception of multiple receive beams in response to each tracking transmission, data for a plurality of laterally spaced locations may be received simultaneously. The entire region of interest 22 is scanned for each receive event by receiving along all the scan lines of the region of interest 22 in response to each transmit event. The monitoring is performed for any number of scan lines. For example, four, eight, sixteen, or thirty-two receive beams are formed in response to each transmission. In yet other embodiments, different transmit events and corresponding receive scan lines are scanned in sequence to cover the entire ROI.

The ultrasound scanner receives a sequence of receive signals. The reception is interleaved with the transmission of the sequence. For each transmit event, a receive event occurs. The receive event is a continuous interval for receiving echoes from the depth or depths of interest. After the transducer completes generation of acoustic energy for a given tracking transmission, the transducer is used for reception of the responsive echoes. The transducer is then used to repeat another transmit and receive event pair for the same spatial location or locations, providing the interleaving (e.g., transmit, receive, transmit, receive, . . . ) to track the tissue response over time. The scanning of the region of interest with ultrasound is repetitive to acquire ultrasound data representing the tissue response at locations of the region of interest at different times while the shear wave propagates through the region of interest. Each repetition monitors the same region or locations for determining tissue response for those locations. Any number of repetitions may be used, such as repeating about 50-100 times. The repetitions occur as frequently as possible while the tissue recovers from the stress, but without interfering with reception.

In one embodiment, receive scan lines at different orientations are used for tracking. At each location, two or more receive beams are formed where the beams are at different angles at the sample location. The transmit scan lines for tracking are at the angle or different angles than one, both, or all the receive scan lines.

The same acoustic echoes are receive beamformed along the scan lines at different angles or orientations. Alternatively, two different scan patterns providing the receive scan lines at different angles are sequentially used in tracking, resulting in different acoustic echoes being beamformed at the different angles. The scan line pattern has two or more scan lines intersecting each or some sample locations at different angles. As a result, the displacements determined along the receive lines intersecting the locations at different angles are subject to different components of the three-dimensional displacement caused by the shear wave. Any difference in receive scan line angles at a given location may be used, such as 90 degrees. Lesser angles may be used due to depth of scanning, directionality of the transducer array, and/or width of the transducer array.

In act 16, the ultrasound scanner estimates a shear wave characteristic for each location in the region of interest 22. The data received by tracking in act 14 is used to detect displacements as a function of time for each location in the region. A maximum or other displacement information over time, arrival time (e.g., time of maximum), and/or the locations are used to estimate the shear wave characteristic.

Tissue motion is detected as a displacement in one, two, or three dimensions. Motion responsive to the generated shear waves is detected from the received tracking or ultrasound data output from act 14. By repeating the transmitting of the ultrasound pulses and the receiving of the ultrasound echoes over the time, the displacements over the time are determined. The tissue motion is detected at different times. The different times correspond to the different tracking scans (i.e., transmit and receive event pairs).

Tissue motion is detected by estimating displacement relative to the reference tissue information. For example, the displacement of tissue along scan lines is determined. The displacement may be measured from tissue data, such as B-mode ultrasound data, but flow (e.g., velocity) or beamformer output information prior to detection (e.g., in-phase and quadrature (IQ) data) may be used.

As the tissue being imaged along the scan lines deforms, the B-mode intensity or other ultrasound data may vary. Correlation, cross-correlation, phase shift estimation, minimum sum of absolute differences or other similarity measure is used to determine the displacement between scans (e.g., between the reference and the current scan). For example, each IQ data pair is correlated to its corresponding reference to obtain the displacement. Data representing a plurality of spatial locations is correlated with the reference data. As another example, data from a plurality of spatial locations (e.g., along the scan lines) is correlated as a function of time. For each depth or spatial location, a correlation over a plurality of depths or spatial locations (e.g., kernel of 64 depths with the center depth being the point for which the profile is calculated) is performed. The spatial offset with the highest or sufficient correlation at a given time indicates the amount of displacement. For each location, the displacement as a function of time is determined. Two or three-dimensional displacement in space may be used. One-dimensional displacement along scan lines or along a direction different from the scan lines or beams may be used.

For a given time or repetition of the scanning, the displacements at different locations are determined. The locations are distributed in one, two, or three dimensions. For example, displacements at different laterally spaced locations are determined from averages of displacements of different depths in the ROI. In another example, displacements are determined for different laterally spaced and range spaced (i.e., depth) locations.

In other embodiments, the displacement as a function of location is determined. Different locations have the same or different displacement amplitude. These profiles of displacement as a function of location are determined for different times, such as for each repetition of transmit/receive events in the scanning of act 14. Line fitting or interpolation may be used to determine displacement at other locations and/or other times.

The displacements for shear data are responsive to the shear wave generated. Due to the origin location of the shear wave and the relative timing of the scanning for displacement, any given location at any given time may be subject to no shear wave-caused displacement or displacement caused by the shear wave.

The ultrasound scanner calculates the shear wave characteristic for each location from the displacements. Any characteristic may be estimated, such as speed or velocity of the shear wave in the tissue. The shear wave speed of the tissue is a velocity of the shear waves passing through the tissue. Different tissues have different shear wave speed. A same tissue with different elasticity and/or stiffness has different shear wave speed. Other viscoelastic characteristics of tissue may result in different shear wave speed. The shear wave speed is calculated based on the amount of time between the pushing pulse and the time of maximum displacement and based on the distance between the ARFI focal location and the location of the displacements. Other approaches may be used, such as determining relative phasing of the displacement profiles.

Other shear wave characteristics of the tissue may be estimated from the location, displacements, and/or timing. The magnitude of the peak displacement normalized for attenuation, time to reach the peak displacement, Young's modulus, or other elasticity values may be estimated. Any viscoelastic information may be estimated as the shear wave characteristic in the tissue.

In act 18 of FIG. 1, the image processor generates an image of a characteristic of the tissue of the patient from results of the estimation. The characteristic is the shear wave characteristic. For example, the image is of shear wave velocity in the tissue.

The estimation provides values for the shear wave characteristic for each location in the region of interest. The locations are distributed in one, two, or three dimensions. The image is of the shear wave characteristic over the one, two, or three dimensions. For example, a shear wave velocity image is generated. For each location, the pixel of the image is modulated by the value of the characteristic. Brightness, color, or other modulation may be used. The shear wave image is displayed alone or overlaid on a B-mode or other ultrasound image.

In additional or alternative embodiments, the output is a graph or alphanumeric text of the shear wave speed for a location or across locations. The image is of alphanumeric text (e.g., “1.36 m/s”) or overlaid as an annotation on a B-mode or flow-mode image of the tissue. A graph, table, or chart of velocity or velocities may be output as the image.

Since the angle based on the tissue orientation is used for the ARFI transmit scan line and/or the tracking scanning, the estimated shear wave characteristic and resulting image may be more accurate. Due to tissue anisotropy, the shear wave propagates along the orientation of the tissue or different than horizontal relative to the transducer array (i.e., top of image) even where the ARFI transmit beam is vertical. By setting the angle, the resulting shear wave estimates are more likely a true measure of the characteristic. Rather than measuring displacements subject to a component of the shear wave caused displacement, the displacements along the direction of the maximum displacement are measured. In alternative embodiments, the angle is used for angle correction of the estimates without altering the transmit or receive scan lines.

In another improvement used with or without the angle of act 11 and corresponding angle of the ARFI transmit scan line 20, the image is generated to represent the direction of propagation of the shear wave in the tissue of the patient. The direction of propagation may be imaged alone or is used for an overlay on the shear wave image (e.g., on the shear wave velocity and B-mode image).

The direction of propagation is indicated by one or more graphics. For example, one or more arrows are added on the image (e.g., in the region of interest) or adjacent to the image. A single vector or direction is determined and used for one or more added arrows. In other embodiments, the direction is determined for two or more locations in the region of interest and corresponding graphics are overlaid to represent the directions at the different locations.

Any graphic may be used. FIG. 3 shows arrows 30. A vector field is displayed as arrows 30. Gradient lines, lines without arrows, video showing movement of an object, or other graphic may be used to indicate the direction on the display screen. Alternatively, the direction of propagation is indicated by color or intensity modulation, such as adding streaking or a band to the pixels along a line or border in a direction of the propagation.

In act 19, the ultrasound scanner (e.g., image processor) determines direction of propagation of the shear wave from the data of the scanning of act 14 and/or the estimates of act 16. In one embodiment, the direction is determined from a gradient of arrival time of the shear wave at a location. The gradient may be determined at different directions to provide a vector field. Alternatively, the gradient is determined for one location, or an average is determined from gradients of multiple locations.

The arrival time is based on the displacements. For example, a time of occurrence of the maximum displacement of the profile of displacements over time is the arrival time. In other embodiments, the first instance after the displacement exceeds a threshold indicates the arrival time of the shear wave. The arrival time map (i.e., spatial distribution of arrival times at the locations in the region of interest 22) represents the time-to-peak or arrive time of the displacements. The gradient along two or three dimensions of the times is calculated. The magnitude of the time gradient represents the speed or velocity of the shear wave. The direction of the gradient represents the direction of propagation. The direction alone is shown, such as showing direction by location or group of locations. The length of the arrows is default. Alternatively, the length, breadth, or color of the arrow or arrows represents the magnitude of the vector or vectors.

In another embodiment, the direction is determined from the displacements at different receive scan line angles. The magnitudes of displacements for the same or similar times along different receive scan line angles relative to the same location provides components of the displacement in two or three dimensions. The vector or vector field is based on two or more displacement maps (e.g., three or more for three-dimensional scanning). By tracking the on-axis displacement of the shear wave using tracking beams of two (or more) different angles, two (or more) displacement maps are provided. Using the angles of the tracking beams to each other and the magnitude of displacement, the vectors for the different locations are determined. Alternatively, a single vector for one location or based on an average for multiple locations is determined. The displacements along different directions are used to provide the components (e.g., axial and lateral components in two dimensions) of displacement. The direction of the vector indicates the propagation direction of the shear wave. The magnitude of the vector represents the magnitude of the displacement.

One or more vectors are determined from displacements along the different orientations for each of one or more locations. The length and direction of the vector or vectors correspond to the magnitude and direction of the tissue displacement due to shear wave propagation. Direction alone may be used. A shear wave displacement vector field or a single displacement vector is determined and displayed.

FIG. 4 shows one embodiment of a system for shear wave imaging with ultrasound. The shear wave images are formed by setting the angle of the pushing pulse and/or tracking based on orientation of tissue of the patient and/or by including indication of detected shear wave propagation direction. The system implements the method of FIG. 1 or other methods.

The system is a medical diagnostic ultrasound imaging system or ultrasound scanner. In alternative embodiments, the system is a personal computer, workstation, PACS station, or other arrangement at a same location or distributed over a network for real-time or post acquisition imaging, so may not include the beamformers 40, 14 and transducer 41.

The system includes a transmit beamformer 40, a transducer 41, a receive beamformer 42, an image processor 43, a display 45, and a memory 44. Additional, different or fewer components may be provided. For example, a user input is provided for manual or assisted selection of angle, display maps, selection of tissue properties to be determined, region of interest selection, selection of direction graphic, and/or other control.

The transmit beamformer 40 is an ultrasound transmitter, memory, pulser, analog circuit, digital circuit, or combinations thereof. The transmit beamformer 40 is configurable to generate waveforms for a plurality of channels with different or relative amplitudes, delays, and/or phasing. The waveforms are relatively delayed and/or phased to steer acoustic beams to focal locations from a selected origin on the transducer 41. Upon transmission of acoustic waves from the transducer 41 in response to the generated electrical waves, one or more beams are formed along one or more transmit scan lines. The transmit beams are formed at different energy or amplitude levels. Amplifiers for each channel and/or aperture size control the amplitude of the transmitted beam.

The transmit beamformer 40 is configured to transmit pulses. The transmit beamformer 40 generates ARFI transmissions and tracking transmissions. A beamformer controller, the beamformer 40, the image processor 43, and/or a sequence loaded from memory 44 sets the sequence of ARFI beam and tracking beams. The ARFI and/or tracking beams are along a scan line or scan lines in any format. The scan lines may be angled relative to a region of interest and/or orientation of tissue. The angle is selectable, such as being set based on user input and/or image processing. A beamformer controller sets the origin and direction of the scan line, providing the angle of the ARFI scan line to the sample location and/or transducer 41.

For tracking tissue displacements, a sequence of transmit beams covering the region of interest is generated. The sequences of transmit beams are generated to scan a two or three-dimensional region. Sector, vector, linear, or other scan formats may be used. The transmit scan lines for tracking are at a same angle to the transducer and/or sample locations as the ARFI transmit scan line (i.e., parallel). Some or all the transmit scan lines for tracking may be at a different angle than the ARFI transmit scan line. The transmit beamformer 40 may generate a plane wave or diverging wave for more rapid scanning.

The ARFI transmit beams may have greater amplitudes than for imaging or detecting tissue motion. Alternatively or additionally, the number of cycles in the ARFI pulse or waveform used is typically greater than the pulse used for tracking (e.g., 100 or more cycles for ARFI and 1-6 cycles for tracking). Aperture differences may be used.

The transducer 41 is a 1-, 1.25-, 1.5-, 1.75-, or 2-dimensional array of piezoelectric or capacitive membrane elements. The transducer 41 includes a plurality of elements for transducing between acoustic and electrical energies. Receive signals are generated in response to ultrasound energy (echoes) impinging on the elements of the transducer. The elements connect with channels of the transmit and receive beamformers 40, 42.

The transmit beamformer 40 and receive beamformer 42 connect with the same elements of the transducer 41 through a transmit/receive switch or multiplexer. The elements are shared for both transmit and receive events. One or more elements may not be shared, such as where the transmit and receive apertures are different (only overlap or use entirely different elements).

The receive beamformer 42 includes a plurality of channels with amplifiers, delays, and/or phase rotators, and one or more summers. Each channel connects with one or more transducer elements. The receive beamformer 42 applies relative delays, phases, and/or apodization to form one or more receive beams in response to a transmission. In alternative embodiments, the receive beamformer 42 is a processor for generating samples using Fourier or other transforms. The receive beamformer 42 may include channels for parallel receive beamforming, such as forming two or more receive beams in response to each transmit event. The receive beamformer 42 outputs beam summed data, such as IQ or radio frequency values, for each beam.

The receive beamformer 42 operates during gaps in the sequence of transmit events for tracking. By interleaving receipt of signals with the tracking transmit pulses, a sequence of receive beams are formed in response to the sequence of transmit beams. After each tracking transmit pulse and before the next tracking transmit pulse, the receive beamformer 42 receives signals from acoustic echoes. Dead time during which receive and transmit operations do not occur may be interleaved to allow for reverberation reduction.

The receive scan lines are at a same angle as the transmit scan lines for tracking but may be at other angles. For example, the receive scan lines are set to be perpendicular to the orientation of the tissue. One or more receive scan lines in a scan format may be at other angles or different angles to other of the receive lines. In one embodiment, the parallel receive beamformation is used to form receive beams that intersect at a sample location in the region of interest, but are not parallel (i.e., are at different angles at the location of intersection). Intersecting receive scan lines may be used for other locations.

The receive beamformer 42 outputs beam summed data representing spatial locations at a given time. Data for different lateral locations (e.g., azimuth spaced sampling locations along different receive scan lines), locations along a line in depth, locations for an area, or locations for a volume are output. Dynamic focusing may be provided. The data may be for different purposes. For example, different scans are performed for B-mode or tissue data than for shear wave velocity estimation. Data received for B-mode or other imaging may be used for estimation of the shear wave velocity. The shear wave at locations spaced from the foci of the pushing pulses are monitored to determine velocity of the shear waves using coherent interference of the shear waves.

The receive beamformer 42 outputs tracking data representing the tissue before, after, and/or during passing of a shear wave. Tracking data is provided to track each sequential shear wave. The tracking data is output for different periods corresponding to the different ARFI transmissions.

The image processor 43 is a B-mode detector, Doppler detector, pulsed wave Doppler detector, correlation processor, Fourier transform processor, application specific integrated circuit, general processor, control processor, image processor, field programmable gate array, digital signal processor, analog circuit, digital circuit, server, group of processors, combinations thereof, or other now known or later developed device for detecting and processing information for display from beamformed ultrasound samples. In one embodiment, the image processor 43 includes one or more detectors and a separate processor for image processing. The image processor 43 may be one or more devices. Multi-processing, parallel processing, or processing by sequential devices may be used.

The image processor 43 performs any combination of one or more of the acts 16-19 shown in FIG. 1. The image processor 43 may control the transmit and/or receive beamformers 40, 42. Beamformed samples or ultrasound data is received from the receive beamformer 42. The image processor 43 is configured by software, hardware, and/or firmware.

The image processor 43 is configured to detect displacements of tissue responding to an ARFI generated shear wave. The detection is from beamformed samples or detected data (e.g., B-mode or Doppler detection) from the beamformed samples. Using correlation, other measure of similarity, or another technique, the movement of tissue relative to a reference is determined from the ultrasound data. By spatially offsetting a tracking set of data relative to a reference set of data in one, two, or three-dimensional space, the offset with the greatest similarity indicates the displacement of the tissue. The processor 43 detects displacement for each time and location. Some of the detected displacements may have magnitudes responsive to a passing shear wave or shear waves.

The image processor 43 is configured to determine a velocity or other shear wave characteristic of shear in the tissue. The determination is based on the signals from tracking the tissue responding to the shear waves created by an ARFI. The signals are used to detect the displacements. To determine the velocity, the displacements are used. The time to reach a maximum displacement and distance from the ARFI focal location provide the velocity. Relative phasing of displacements over time of different locations or other approaches may be used to determine velocity.

The image processor 43 is configured to determine an angle of propagation of the shear wave in the tissue. The shear wave may propagate in general along a line that is not perpendicular to the ARFI transmit beam. The anisotropy of tissue may result in the propagation being greatest along a non-perpendicular line. The image processor 43 uses displacements and/or times of shear wave occurrence to determine a direction of propagation.

The image processor 43 generates display data, such as annotation, graphic overlay, and/or image. The display data is in any format, such as values before mapping, gray scale or color-mapped values, red-green-blue (RGB) values, scan format data, display or Cartesian coordinate format data, or other data. The display data may be a shear wave images, such as a shear wave velocity image using color coding for velocities. The display data may be a graphic indicating direction and/or magnitude of shear wave propagation. Combinations of graphics for vector imaging and shear wave velocity imaging may be used, such as represented in FIG. 3.

The processor 43 outputs velocity information appropriate for the display device 20, configuring the display device 20. Outputs to other devices may be used, such as outputting to the memory 44 for storage, output to another memory (e.g., patient medical record database), and/or transfer over a network to another device (e.g., a user computer or server).

The display device 20 is a CRT, LCD, projector, plasma, printer, or other display for displaying shear velocity, graphics, user interface, validation indication, two-dimensional images, or three-dimensional representations. The display device 20 displays ultrasound images, the velocity, and/or other information. For example, the display screen outputs tissue response information, such as a one, two, or three-dimensional distribution of the velocity or other shear wave characteristic. Velocities or shear wave characteristics for different spatial locations form an image. The velocities or characteristics represented in the image may more accurately reflect the shear wave response of tissue due to use of the transmit and/or receive angle oriented based on tissue orientation.

A graphic, such as one or more arrows, may be overlaid or displayed adjacent to the shear wave image to display the detected propagation direction. Other images may be output as well, such as overlaying the velocity as a color-coded modulation for a region of interest on a gray scale B-mode image with or without vector representation for the angle of propagation as detected.

In one embodiment, the display device 20 outputs an image of a region of the patient, such as a two-dimensional Doppler tissue or B-mode image. The image includes a location indicator for the velocity. The location indicator designates the imaged tissue for which a velocity value is calculated. The velocity is provided as an alphanumeric value on or adjacent the image of the region. The image may be of the alphanumeric value with or without spatial representation of the patient. A graphic for the propagation angle may be output to assist in understanding the velocity value for diagnosis.

The processor 43 operates pursuant to instructions stored in the memory 44 or another memory. The memory 44 is a computer readable storage media. The instructions for implementing the processes, methods and/or techniques discussed herein are provided on the computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

The memory 44 alternatively or additionally stores data used in estimation of shear wave characteristic, setting of an angle, and/or detection of shear wave propagation angle. For example, the transmit sequences and/or beamformer parameters for ARFI and tracking, including the angle or beamformer settings to implement the angle, are stored. As another example, the region of interest, received signals, detected displacements, estimated shear wave characteristic values, detected vector or vectors, graphics, and/or display values are stored.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method for shear wave imaging with an ultrasound scanner, the method comprising:
positioning a region of interest for tissue of a patient;
receiving an angle;
transmitting a radiation force pulse from a transducer of the ultrasound scanner to a focus location in or by the region of interest of the tissue of the patient, the radiation force pulse transmitting to intersect the focus location at the angle, a shear wave being generated due to the radiation force pulse;
scanning, by the ultrasound scanner, the region of interest with ultrasound as the shear wave propagates in the region of interest;
estimating a shear wave characteristic from the scanning;
generating an image of the shear wave characteristic of the tissue of the patient.

2. The method of claim 1 wherein positioning the region of interest comprises positioning the region of interest on an ultrasound image by user input of the region of interest.

3. The method of claim 1 wherein receiving the angle comprises receiving user input of the angle.

4. The method of claim 1 wherein receiving the angle comprises determining the angle from image processing and without user input of the angle.

5. The method of claim 4 wherein determining the angle comprises determining from a vector field based on arrival time.

6. The method of claim 4 wherein determining the angle comprises determining from a vector field based on displacements along non-parallel receive scan lines.

7. The method of claim 1 wherein receiving the angle comprises determining an orientation of anatomy within the region of interest and setting the angle to be perpendicular to the orientation.

8. The method of claim 7 wherein determining the orientation comprises determining the orientation as a direction of muscle or collagen fibers.

9. The method of claim 1 wherein transmitting comprises forming an acoustic beam focused at the focus location and being along a transmit scan line, the transmit scan line being at the angle.

10. The method of claim 9 wherein the region of interest is rectangular or square and wherein the transmit scan line is non-perpendicular and non-parallel to all sides of the rectangular or square region of interest.

11. The method of claim 1 wherein scanning comprises repetitively transmitting tracking pulses over the region of interest and receiving acoustic responses responsive to the tracking pulses.

12. The method of claim 1 wherein estimating the shear wave characteristic comprises estimating shear wave velocity and wherein generating the image comprises generating a shear wave image.

13. The method of claim 1 wherein generating the image comprises determining vectors from gradients of arrive times of the shear wave at locations in the region of interest and generating the image as representing the vectors.

14. The method of claim 1 wherein scanning comprises scanning with receive scan lines at different orientations, and wherein generating the image comprises determining vectors from displacements along the different orientations, and generating the image as representing the vectors.

15. A method for shear wave imaging with an ultrasound scanner, the method comprising:
transmitting a radiation force pulse from a transducer of the ultrasound scanner to tissue of a patient, a shear wave being generated due to the radiation force pulse;
scanning, by the ultrasound scanner, the tissue with ultrasound as the shear wave propagates in the tissue;
determining a direction of propagation of the shear wave from the scanning; and
generating an image representing the direction of propagation of the shear wave in the tissue of the patient.

16. The method of claim 15 wherein scanning comprises determining displacements over time caused by the shear wave for each of a plurality of locations in the tissue, and wherein determining the direction comprises determining the direction from a gradient of arrival time of the shear wave at the location, the arrival time being based on the displacements.

17. The method of claim 15 wherein scanning comprises determining displacements over time caused by the shear wave for each of a plurality of locations in the tissue, the displacements determined along receive lines intersecting the locations at different angles, and wherein determining the direction comprises determining the direction from the displacements at the different angles.

18. The method of claim 15 wherein generating the image comprises generating a vector field as arrows showing the direction by location in a region of interest.

19. A system for shear wave imaging with ultrasound, the system comprising:
a transmit beamformer configured to transmit a pushing pulse along a transmit line into tissue of a patient, a transmit angle of the transmit line of the pushing pulse relative to a location in the tissue being selectable;
a receive beamformer configured to receive signals from scanning after the transmission of the pushing pulse;
an image processor configured to determine, from the receive signals, shear wave velocity and a propagation angle of a shear wave in the tissue; and
a display configured to output a shear velocity image of the shear wave velocity with a graphic representing the propagation angle.

20. The system of claim 19 wherein the graphic comprises an arrow.
